(19) United States
(12) Patent Application Publication
(10) Pub. No.: US 2011/0264180 A1
(43) Pub. Date: Oct. 27, 2011

(54) SPINAL CORD STIMULATOR LEAD ANCHOR

(76) Inventor: Dennison R. Hamilton, Leawood, KS (US)

(21) Appl. No.: 12/768,053

(22) Filed: Apr. 27, 2010

(51) Int. Cl. A61N 1/05

(52) U.S. Cl. 607/117

(57) ABSTRACT

The lead of a spinal cord stimulator implanted along the spinal cord of a recipient is secured by an anchor staple at a selected location. The anchor staple comprises a loop that receives and holds the lead at the selected location along the spine. A pair of spaced legs extend from the loop and present an initially open configuration for receiving an anchor point presented by bone, ligament, fascia or skin. The legs are closed to engage the anchor point and thereby positively secure the lead to prevent migration of the implanted lead. Several staples may be applied in a row to assure optimal positioning of the lead.

This invention relates to the treatment of back pain by an implanted spinal cord stimulator and, in particular, to the prevention of the adverse affect of a lead migration which negates the effectiveness of the treatment.

### BACKGROUND OF THE INVENTION

It has been estimated that eighty percent of the population of the United States will suffer from back pain at some time in their life. A portion of the affected population will suffer from intractable back pain that is unresponsive to surgical intervention, injection therapy, physical therapy or narcotic analgesics. Those unresponsive to conventional treatment are candidates for a spinal cord stimulator which is implanted in a manner similar to a heart pacemaker. Leads with electrodes are placed along the spinal cord in the epidural space and are energized by an implantable pulse generator which sends an electrical impulse through the leads and electrodes over the spinal cord to produce a sense of numbness or tingling where the person is experiencing pain.

In addition to the high cost of this technology, there are risks of adverse consequences. A significant adverse event is lead migration which may occur approximately twenty percent of the time. Correction of the migration may be expensive and challenging because of the amount of scar tissue that forms around the leads themselves.

A standard procedure is to implant the leads along the spinal cord and affix them to bone, ligament, fascia or skin using suture material applied directly to the lead or over a suture sleeve. This anchors the lead to prevent migration but, in practice, the process of tying the leads to the supportive tissues can cause lead migration. Well placed sutures, if not firmly tight, will allow migration as the person moves during activities of daily living.

### SUMMARY OF THE INVENTION

In an embodiment of the present invention, an anchor staple receives and holds an implanted lead at a selected location along the spine. The staple presents a loop to accommodate the lead or suture sleeve, and is closed to engage bone, ligament, fascia or skin to apply a constant pressure to secure the lead and thereby prevent, or significantly minimize migration. The staples may be secured at spaced locations, and applied over the lead or suture sleeve with a clip applicator. A pair of spaced legs extending from the loop have an initially open configuration for receiving bone, ligament, fascia or skin at the selected location, and are movable to a final, closed position engaging the bone, ligament, fascia or skin to secure the lead.

In another aspect of the invention, the loop of the staple and the legs thereof are of disposed substantially in a common plane, and are closed about the bone, ligament, fascia or skin to secure the lead in place.

In another aspect of the present invention, the loop is provided with inwardly extending serrations for securing the lead therein and precluding migration of the implanted lead.

In yet another aspect of the present invention, each of the legs of the anchor staple has a generally C-shaped configuration and the legs are spaced apart in opposed relationship to present an initially open space therebetween for receiving the bone, ligament, fascia or skin, each leg having a distal end presenting an inward projection for engaging the bone, ligament, fascia or skin when the loop is closed to secure the lead.

A further aspect of the present invention comprises an anchor staple configuration wherein the legs initially extend outwardly away from each other and present opposed, inwardly projecting end portions movable inwardly to a closed position to secure the staple at the selected location.

Additionally, a method of implanting the lead of a spinal cord stimulator comprises the steps of providing an anchor staple having a loop presenting an opening for receiving the lead of a spinal cord stimulator, and a pair of spaced legs extending from said loop and having an initially open configuration. The lead is received within the loop at a desired location, the legs of the loop are positioned about bone, ligament, fascia or skin over the spinal column, and then closed on the structure to secure the lead thereto to prevent migration. In addition, forceps may be provided having jaws for receiving and applying the staple, and a recess within the jaws for receiving the loop to prevent flattening of the loop when the jaws are closed.

Other advantages of this invention will become apparent from the following description taken in connection with the accompanying drawings, wherein is set forth by way of illustration and example, an embodiment of the present invention.

### BRIEF DESCRIPTION OF THE DRAWINGS

FIG. 1 is a plan view of an embodiment of the anchor staple of the present invention showing a pair of spaced legs thereof in an initially open configuration.

FIG. 2 is a view similar to FIG. 1, showing the legs in a final, closed position.

FIG. 3 is a view similar to FIG. 2, but showing the loop lying generally in a plane diverging from the plane of the legs at an acute angle.

FIG. 4 shows an implanted lead of the spinal cord stimulator secured by the anchor staple.

FIG. 5 is a plan view of an alternative embodiment of the staple in an open condition.

FIG. 6 is a view similar to FIG. 5, but showing the staple closed.

FIG. 7 is a perspective view of the staple of FIG. 6 showing the loop thereof forming an acute angle with the legs thereof.

FIG. 8 is a simplified, fragmentary illustration showing attachment of the staple of FIG. 3 to an anchor point presented by bone, ligament, fascia or skin.

FIG. 9 is a view similar to FIG. 8, showing attachment of the anchor staple of FIG. 7.

FIG. 10 is a fragmentary, longitudinal cross-section of the spine showing the lead of a spinal cord stimulator inserted in the epidural space along the spinal cord, and secured to bone structure by the anchoring staple of the present invention.

FIG. 11 is a transverse cross-sectional view of the spinal column showing the loop of the anchor staple receiving and holding the lead, and the legs thereof in a closed position engaging an anchor presented by the spinous process.

FIG. 12 is a perspective view of forceps which may be used in the installation of the anchor staples of the present invention.

### DETAILED DESCRIPTION

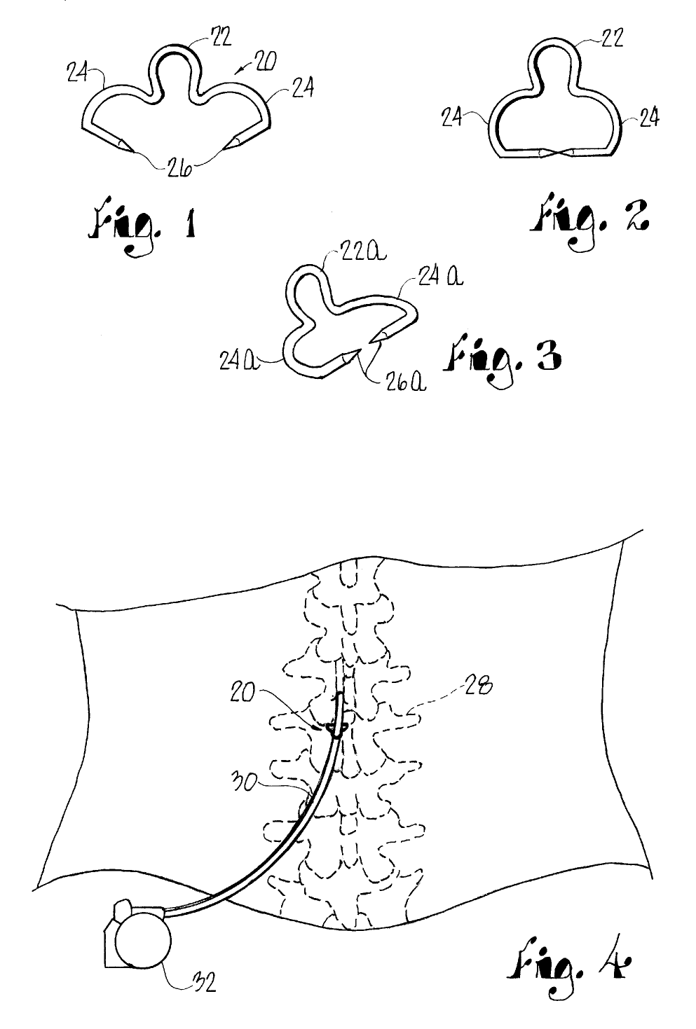

Referring initially to FIGS. 1 and 2, an anchor staple 20 of the present invention is shown open prior to application in FIG. 1 and in a closed condition in FIG. 2 and preferably comprises a titanium rod having a central loop 22 and a pair of spaced, opposed legs 24 of a generally C-shaped configuration presenting spaced, pointed distal ends 26 initially presenting an opening therebetween. In use, as will be discussed hereinbelow, the legs 24 are closed as shown in FIG. 2 to engage and embrace an anchor point adjacent the spine presented by bone, ligament, fascia or skin at the location under treatment. This is illustrated in FIG. 4 where the spinal column 28 of a patient is shown in broken lines. A lead 30 extending from an implanted pulse generator illustrated at 32 is secured by the staple 20. Depending upon the particular installation, as shown in FIG. 3 the plane of the loop 22a may be disposed at an acute angle to the plane presented by the opposed legs 24a, 26a at an angle, for example, not exceeding approximately 80 degrees.

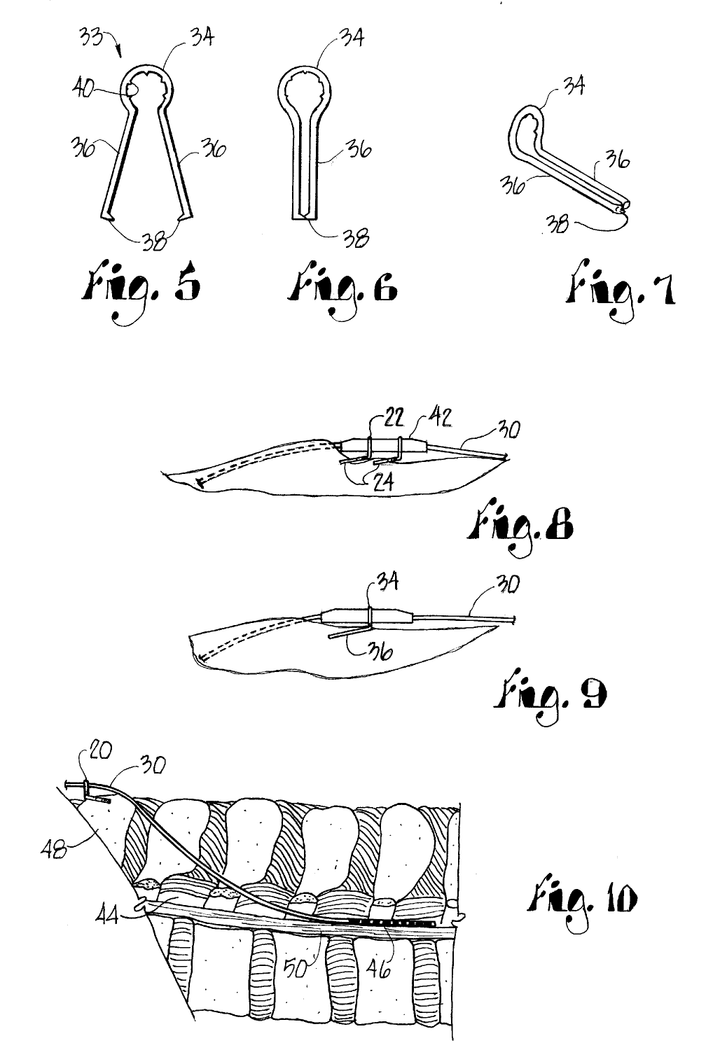

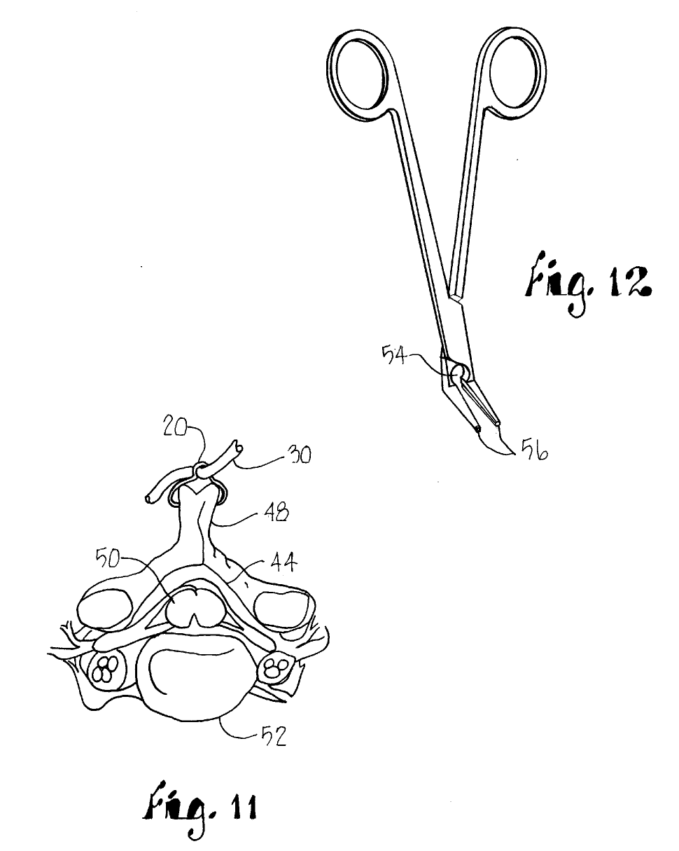

A second embodiment 33 of the anchor staple of the present invention is shown in FIGS. 5-7 and comprises a loop 34 having a pair of straight legs 36 extending therefrom and terminating in inwardly directed projections 38 respectively. If desired, the interior of the loop 34 may be provided with radially inwardly extending serrations 40 for gripping a sheath 42 on the lead 30 where the loop 34 is located (FIGS. 8 and 9). FIGS. 8 and 9 illustrate the staples of FIGS. 3 and 7, respectively, attached to bone to anchor and hold the lead 30 as will be discussed more fully hereinbelow.

FIG. 10 shows a portion of the spine in cross-section where it may be seen that the lead 30 extends inwardly to the epidural space 44 where eight spaced electrodes 46 are exposed. It may be appreciated that typically two leads are inserted to provide sixteen electrodes along the spinal cord, one lead being illustrated herein in FIG. 10 for clarity. Accordingly, the lead 30 contains eight separate insulated leads terminating in the respective electrodes 46. In the present invention, any movement or displacement of the lead 30 is precluded by the anchor staple 20 or 33 which embraces and grips the lead 30 and secures it to suitable bone, ligament, fascia or skin, and specifically a spinous process 48 in the illustration of FIG. 10. It will be appreciated that a series of staples 20 or 33 may be spaced along the lead 30 as needed in a particular treatment, as illustrated in FIG. 8.

FIG. 11 is a transverse cross-section through the spine illustrating the placement of the anchor staple 20 in FIG. 10. The spinal cord is illustrated at 50 within the epidural space 44 and on the posterior side of the vertebral body 52. Accordingly, by a comparison of FIGS. 10 and 11, it may be seen that the lead 30 of the present invention may be secured to the spinous process 48, directed inwardly to the epidural space 44 and then along the spinal column 28 to position the electrodes 46 at the desired location to alleviate the pain experienced by the patient. Although only one series of electrodes 46 is illustrated herein, it will be appreciated that electrodes may be spaced along the lead 30 as necessary to provide the relief required in a specific case. Furthermore, several anchor staples 20 or 33 may be applied in a row to assure optimal positioning of the lead 30. As lead migration is minimized by utilization of the anchoring staples of the present invention, the patient may enjoy normal movement and activity without displacing the stimulator lead and undergoing undesired additional surgical procedures.

Forceps utilized in installing the staple of the present invention are illustrated in FIG. 12, and are characterized by a circular opening 54 spaced inwardly from the jaws 56 of the forceps to present an opening for receiving the essentially round loop 22 or 34 of the staples of FIGS. 1-7 to preclude deformation of the loop when forceps are used to close the legs of the staple. The forceps may be advantageously utilized with both embodiments to close the staple to the configurations shown in FIGS. 2, 3, 6 and 7 without distorting the loop 22 or 34 which embraces the stimulator lead.

It should be understood that while certain forms of this invention have been illustrated and described, it is not limited thereto except insofar as such limitations are included in the following claims.

Having thus described the invention, what is claimed as new and desired to be secured by Letters Patent is:

1. In a spinal cord stimulator:
a lead adapted to be implanted along the spinal cord of a recipient and having a plurality of conductors connected to a corresponding plurality of electrodes spaced along said lead, and
an anchor staple on said lead for securing the lead to the recipient at a selected location along the spine,
said staple comprising a loop presenting an opening for receiving and holding said lead at said selected location along the spine, and a pair of spaced legs extending from said loop and having an initially open configuration for receiving an anchor point presented by bone, ligament, fascia or skin adjacent said location, and moveable to a final, closed position engaging said anchor point to secure said lead, whereby to prevent migration of the lead implanted in a recipient.

2. The spinal cord stimulator as claimed in claim 1, wherein said loop and said legs are disposed substantially in a common plane.

3. The spinal cord stimulator as claimed in claim 1, wherein said loop has inwardly extending serrations.

4. The spinal cord stimulator as claimed in claim 1, wherein each of said legs has a generally C-shaped configuration and is initially open to present a space therebetween for receiving said anchor point.

5. The spinal cord stimulator as claimed in claim 1, wherein each of said legs has a distal end presenting an inward projection for engaging said anchor point.

6. The spinal cord stimulator as claimed in claim 1, wherein said loop lies generally in a plane, and said legs diverge from said plane at an acute angle.

7. The spinal cord stimulator as claimed in claim 1, wherein said legs initially extend outwardly away from each other and present opposed, inwardly projecting end portions movable inwardly to a closed position to secure the staple at said selected location.

8. A method of implanting a lead of a spinal cord stimulator, said method comprising the steps of:
providing an anchor staple having a loop presenting an opening for receiving the lead of the spinal cord stimulator, and a pair of spaced legs extending from said loop and having an initially open configuration,
receiving the lead within said loop at a desired location,
positioning said legs of the loop about an anchor point presented by bone, ligament, fascia or skin over the spinal column, and
closing said legs on said anchor point to secure said lead thereto and thereby prevent migration of the lead of the spinal cord stimulator.

9. The method as claimed in claim 8, wherein said method includes providing forceps having jaws for receiving and applying said staple, and a recess within said jaws for receiving said loop to prevent flattening of the loop when the jaws are closed.
